(19) United States
(12) Patent Application Publication
(10) Pub. No.: US 2025/0213853 A1
(43) Pub. Date: Jul. 3, 2025

(54) Alteration of Blood Pressure and Modulation of Renal Blood Flow using Dorsal Root Ganglion Stimulation

(71) Applicant: Nutan Mytle, Gaithersburg, MD (US)

(72) Inventor: Nutan Mytle, Gaithersburg, MD (US)

(21) Appl. No.: 18/403,651

(22) Filed: Jan. 3, 2024

(51) Int. Cl. A61N 1/05; A61N 1/36

(52) U.S. Cl. CPC A61N 1/0551; A61N 1/36071

(57) ABSTRACT

Blood flow to the kidney primarily comes from the renal arteries which are controlled by the renal plexus. This plexus is innervated by the sympathetic chain and dorsal root ganglion upstream. In this embodiment neuromodulation of dorsal root ganglion between T8 and L3 can be used to alter renal blood flow and, thus, blood pressure. The neuromodulation at at frequencies between 0.01 Hz and 19.99 Hz at these DRG levels can be used to increase blood flow to the kidneys and to treat hypertension.

### CROSS-REFERENCE TO RELATED APPLICATIONS

N/a

### FEDERALLY SPONSORED RESEARCH AND DEVELOPMENT

No federal research grant was used.

### PRIOR ART

Currently dorsal root ganglion (DRG) stimulation is used in interventional pain to modulate nerves that have been injured or inflamed and are causing neuropathic pain. A lead with electrode is placed in proximity to the DRG, which is located outside the dorsal column epidural space and out of the neural foramen. Subsequently the lead electrodes send electric signals to stimulate DRG in order to restore the firing activity back to baseline.

A different technique to modulate blood pressure has the FDA approval. This technique performs radiofrequency ablation of renal plexus nerves around the renal artery in order to prevent kidney from vasoconstricting and thus decreasing the blood pressure. However, during ischemic injury, renal sympathetic denervation can impair the blood flow, which may harm the patient. Furthermore, there is natural sympathetic reinnervation of the plexus which would cause the hypertension to return.

However, there is no neuromodulation procedure to alter blood pressure or modulate renal blood flow using dorsal root ganglion stimulation.

### SUMMARY OF THE INVENTION

Objective 1. In one embodiment, perform dorsal root ganglion stimulation between T8 and L3 spinal levels to lower blood pressure acutely or chronically.

Objective 2. In another embodiment, perform dorsal root ganglion stimulation at any of the electrical frequencies between 0.01 Hz and 19.99 Hz to lower blood pressure acutely or chronically or to modulate renal blood flow.

Objective 3. In another embodiment, perform dorsal root ganglion stimulation between T8 and L3 spinal levels to modulate renal blood flow.

Objective 4. In another embodiment, the dorsal root ganglion stimulation can be turned off to protect the patient from ischemia, which renal sympathetic denervation would not be able to.

Objective 5. In another embodiment, the dorsal root ganglion stimulation is as proximal and upstream that natural sympathetic reinnervation of renal plexus would not be an issue for treating chronic hypertension as it would with renal sympathetic denervation.

### BRIEF DESCRIPTION OF DRAWINGS

FIG. 1. demonstrates the spinal canal and epidural space (cylinder) from which the nerve roots exist on each side. There is one DRG located on each of the nerve roots right outside the neural foramen and the epidural space. After the DRG the nerve root travels to its destination.

FIG. 2. demonstrates the same spinal cord and DRG structure from FIG. 1, but it adds the DRG stimulation leads which are around the dorsal root ganglion. In this embodiment, it sends electrical frequency between 0.01 Hz and 19.99 Hz, inclusive, to stimulate the dorsal root ganglion. Each DRG lead stimulates that side and level of DRG.

FIG. 3. demonstrates that blood to the kidney is supplied by the renal artery, which has renal plexus nerves around the outside of renal artery in circumferential fashion. This renal plexus gets neural directions from the autonomic ganglion and renal sympathetic neurons, such as aorticorenal ganglion or mesenteric ganglions. All of these ganglion that innervate the kidney come from nerve roots between T8 and L3. Hence stimulation of DRG between T8 and L3 nerve roots can modulate the renal plexus around the renal artery.

FIG. 4. shows a flowchart on how stimulating the DRG between T8 and L3 can stimulate the autonomic ganglion, which in turn stimulates the renal plexus around the renal artery. This stimulation leads to modulation of blood pressure, including mean arterial blood pressure, systolic blood pressure and diastolic blood pressure. Furthermore, the stimulation of the renal plexus can lead to modulating renal blood flow to the kidneys.

### DETAILED DESCRIPTION OF THE INVENTION

In one embodiment, a lead or a device that stimulates the dorsal root ganglion through electrical or ultrasound or thermal energy is used for neuromodulation.

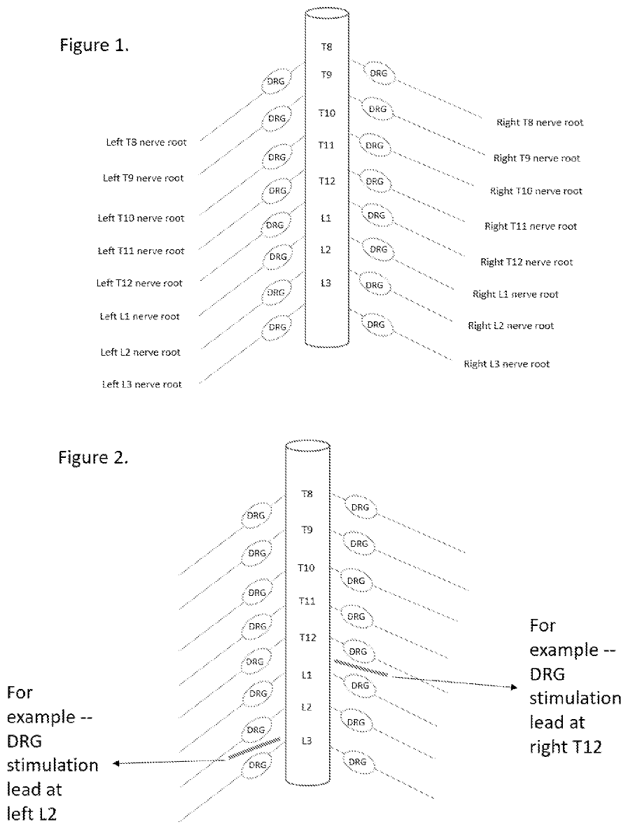

This device is placed to stimulate the dorsal root ganglions at any level between Thoracic level 8 (T8) and Lumbar level 3 (L3), including T8 and L3. The dorsal root ganglions are shown in FIG. 1.

The device can be placed via transforaminal approach or passing out of neural foramen via epidural space or placed surgically near the DRG or field stimulation targeting DRG through skin, tissue, muscles or around the pedicle. An embodiment of lead is shown in FIG. 2 with right T12 or left L2 DRG having a lead around the DRG.

The electrical frequencies used to stimulate the dorsal root ganglion range from 0.01 Hz to 19.99 Hz, both values inclusive.

The ultrasound and thermal energy frequencies will be based on correlation from electrical energy frequencies between 0.01 Hz and 100 Hz.

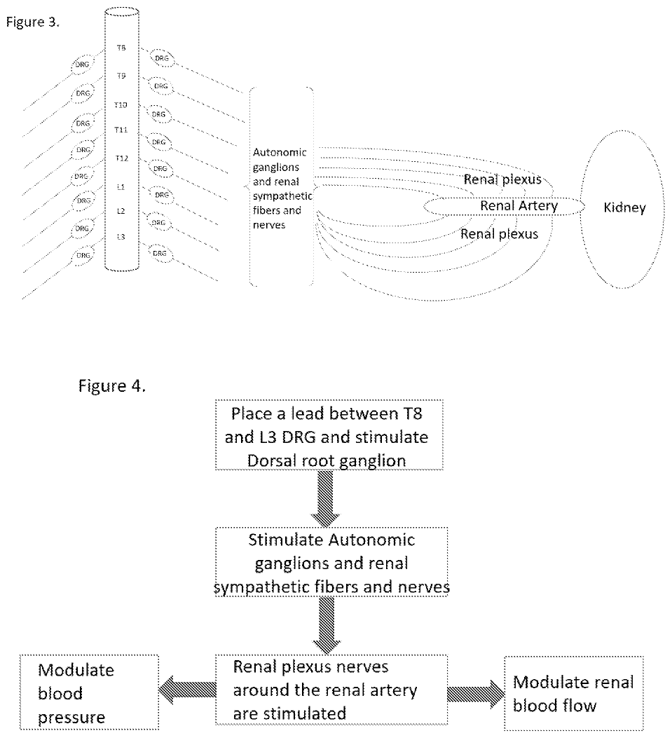

FIG. 3. shows how the renal plexus around the renal artery is innervated by the autonomic ganglion and the sympathetic chains of inferior thoracic and upper lumbar trunks, which in turn are innervated from the dorsal root ganglion from T8 and L3, inclusive.

In one embodiment, the dorsal root ganglion stimulation therapy is used to modulate blood pressures, including mean arterial blood pressure, systolic blood pressure and diastolic blood pressures.

In another embodiment, this DRG therapy can also be used to modulate renal blood flow. Both of these embodiments are shown in a flowchart in FIG. 4.

1. The invention is directed to stimulate the dorsal root ganglion at frequencies between 0.01 Hz and 19.99 Hz, inclusive.

2. The invention is directed to stimulate the dorsal root ganglion between T8 and L3, inclusive, in order to modulate blood pressure.

3. The invention is directed to stimulate the dorsal root ganglion between T8 and L3, inclusive, in order to modulate renal blood flow.
